(19) United States
(12) Patent Application Publication
(10) Pub. No.: US 2023/0102471 A1
(43) Pub. Date: Mar. 30, 2023

(54) Composition And Method for Modulating Hydrogen Ion Physiology Related to Weight Loss and Metabolism

(71) Applicant: Cannogen Biosciences, Inc., Incline Village, NV (US)

(72) Inventor: Edward Larry McCleary, Incline Village, NV (US)

(73) Assignee: Cannogen Biosciences, Inc., Incline Village, NV (US)

(21) Appl. No.: 17/954,325

(22) Filed: Sep. 28, 2022

Related U.S. Application Data
(60) Provisional application No. 63/250,001, filed on Sep. 29, 2021.

(51) Int. Cl. A61K 31/7048; A61K 31/4375; A61K 31/352; A61K 31/05; A61K 31/12; A61K 31/194; A61K 33/00; A61K 31/201; A61K 31/122; A61K 31/202; A61K 31/4412; A61K 33/06; A61K 36/24

(52) U.S. Cl. CPC A61K 31/7048; A61K 31/4375; A61K 31/352; A61K 31/05; A61K 31/12; A61K 31/194; A61K 33/00; A61K 31/201; A61K 31/122; A61K 31/202; A61K 31/4412; A61K 33/06; A61K 36/24

(57) ABSTRACT

A nutritional supplement composition that enhances the incorporation of calcium and other bone building elements in bone and that prevents bone breakdown, specifically due to the chronic metabolic acidosis that the typical Western diet generates each day. The transcription factor NFKB (Nuclear Factor Kappa B), which underlies some of this physiology, has also been shown to adversely affect fat metabolism. The enzymatic protein complex, AMP Kinase (Adenosine Monophosphate Kinase), a key regulator of energy metabolism, has been shown to reverse the effects of excessive NFKB activity on bone and fat metabolism. The novel nutrients modulate nutrient partitioning and fat metabolism in a human or animal so as to increase oxidation of fat, decrease storage of fat and promote increased storage of glycogen. They are composed of one or more of nutrients selected from the group consisting of Hesperidin, Berberine, Epigallocatechin 3-Gallate (EGCG), Resveratrol, Curcumin, Ginsenosides, Gingerols, Hispidulin, Honokiol, Magnolol and Quercetin. A method for modulating nutrient partitioning in a human involves orally or parenterally administering the aforementioned composition to the human or animal, preferably on a daily basis, for a therapeutically effective period of time. Preferably, the method further involves having the recipient follow a specific dietary regimen wherein the glycemic index is less than 60 and the daily calorie consumption from carbohydrates is less than about 50% and the daily calorie consumption from protein is at least about 20%. Optionally, the method further involves an exercise program, a stress reduction program and/or a blood donation program.

### CROSS-REFERENCE TO RELATED APPLICATION

This non-provisional application relies for priority on 63/250,001, entitled “Composition and Method for Modulating Hydrogen Ion Physiology Related to Weight Loss and Metabolism,” filed on 29 Sep. 2021, the entire specification of which is incorporated by reference as if fully set forth herein.

### BACKGROUND OF THE INVENTION

### 1. Field of the Invention

This invention relates in general to nutraceuticals, including functional foods, dietary supplements, nutritional supplements, medical foods, botanical drugs, and drugs and more particularly, relates to nutraceuticals and methods that are useful for supporting and promoting both a healthy calcium and muscle physiology and healthy fat metabolism.

### 2. Background Art

Compositions and methods designed to reduce fat levels or otherwise improve metabolism in the body are well known in the art. Many proposals for food supplements and additives have been made over the years.

A weight loss composition designed to burn and reduce synthesis of fats is disclosed, e.g., in U.S. Pat. No. 5,626,849 to Hastings et al. The composition taught in Hastings, et al. contains chromium, L-carnitine, gamma-linoleic acid, (−) hydroxycitric acid, choline, inositol, antioxidants and herbs. The preferred antioxidants are said to be Coenzyme Q10.

U.S. Pat. No. 6,020,378 to Cook et al. discloses a method for selectively altering body fat levels in animals involving administering to the animal a combination of conjugated linoleic acid isomers in a ratio selected to retain a desirable benefit attributable to one isomer while counteracting an undesirable effect of the same isomer.

European Patent Application No. EP0779033 discloses an edible fat spread which is said to contribute to an improved blood lipid profile. The spread contains triglyceride fat, the fatty acid residues of which includes conjugated linoleic acid (CIA) residues.

U.S. Pat. No. 5,895,652 to Giampapa discloses a method and composition which are said to supply key elements necessary for proper metabolization and function of the human body, wherein the composition includes vitamins, minerals, plant extracts, aminos, neurochemical precursors, enzymes and pH regulating agents.

U.S. Pat. No. 4,599,232 to Bertelli discloses a pharmaceutical composition for treatment of tissue energetic and metabolic disorders, wherein the composition contains carnitine or acetylcarnitine and coenzyme Q10 in ratios from 100:1 to 2:1, together with pharmaceutically acceptable excipients.

U.S. Pat. No. 5,973,004 to Howard discloses a composition for oral or parenteral administration to animals for prevention or treatment of syndromes or diseases arising from dysfunctional energy metabolism. The Howard composition includes a combination of L-carnitine and acetyl-L-carnitine, preferably with pantothenic acid or ubiquinone.

U.S. Pat. No. 6,048,846 to Cochran discloses a composition which is said to fight disease and restore the conditions of the body on a cellular level, wherein the composition contains specific and calculated quantities of hormones, amino acids, amino sugars, coenzymes, enzymes and mineral ions.

German patent no. DE 4304394A1 (abstract) discloses a composition for nourishing oncological patients, composed of fats and optionally proteins and/or carbohydrates, wherein the fat contains oleic acid, linoleic acid, alpha lipoic acid, eicosapentanoic acid and docosahexanoic acid.

WO 89/01740 discloses a composition intended for diet fortification to increase the efficiency of muscle work wherein the composition includes coenzyme Q10 as an essential component, along with nutrients, salts, vitamins, trace substances, flavorings, aromatics, etc.

None of the foregoing references is specifically concerned with the issue of altering the nutrient partitioning in the body so as to increase oxidation of fat and storage of carbohydrate (in the form of glycogen).

Proposals have been made for nutritional supplement compositions to provide for increased oxidation of fat in humans, simultaneously promoting increased storage of glycogen, for example, in U.S. Pat. No. 6,579,866, granted to the inventor of the present invention and entitled “Composition and Method for Modulating Nutrient Partitioning.” Several of the compositions and combinations of nutraceuticals described and claimed therein are found effective for the purposes stated therein. In recent studies, additional such compositions have been found to be as, if not more, effective to produce enhanced results in terms of weight loss and other beneficial effects.

### SUMMARY OF THE INVENTION

Accordingly, one feature of this invention is to provide an improved set of compositions and methods for modulating nutrient partitioning in the body so as to increase oxidation of fat and storage of glycogen. This invention teaches and claims the improved and additionally effective compositions combinations and permutations of one or more of the group of nutrients consisting of Hesperidin, Berberine, Epigallocatechin 3-Gallate, Resveratrol, Curcumin, Ginsenosides, Gingerols, Hispidulin and Quercetin.

A nutritional supplement composition for normalizing impaired or deteriorating fat metabolism and nutrient partitioning in humans and animals provides the functions of: (1) down-regulation of the effects of chronic metabolic acidosis; and (2) inhibition of prostaglandin signaling by modulating the activity of various COX enzymes (cyclo-oxygenase) that further enhances bone breakdown and various adverse effects on fat metabolism. This is done by incorporating into the diet of humans and animals of combinations and permutations of the novel and improved nutrients which address one or more of the functional aspects mentioned above.

Another feature of this invention is to expand and include these nutraceuticals to provide for improved Hydrogen Ion Physiology (HIP) mechanisms, and to incorporate this group of novel nutrients and food supplements related to enhance and augment healthy fat metabolism and nutrient partitioning in a regimen to include these nutrients additionally and in supplement to a healthy diet.

Still another feature of the present invention is to include additional group of nutrients that provide enhanced and effective methods of administering the above described nutraceuticals, together with or in substitution with the nutrients disclosed in the aforementioned U.S. Pat. No. 6,579,866.

### DETAILED DESCRIPTION OF THE PREFERRED EMBODIMENTS

this invention and its novel combination on of nutrient and food supplements relies mainly upon recent discoveries in the field. It has been recently discovered that the RANKL/NFKB signaling pathways play a critical role in osteoclast activation and bone loss.

It has also been shown that the acidosis/COX pathway (as taught in the current HIP (Hydrogen Ion Physiology) patent) activates the RANK/RANKL/NFKB signaling pathway.

Further, it has been shown that the acidosis/COX pathway and the RANK (Receptor Activator of Nuclear Factor Kappa B)/RANKL (Receptor Activator of Nuclear Factor Kappa B Ligand)/NFKB (Nuclear Factor Kappa B) pathways converge and act together to turn on osteoclast activity resulting in enhanced bone loss.

One novel aspect of the invention is that while the acidosis/COX pathway and the RANKL/NFKB pathway independently up-regulate osteoclast activity, they magnify each other's negative biological impact by feeding into the same downstream signaling system. By combining these two approaches, novel multiplicative benefits accrue because both metabolic abnormalities would be down-regulated simultaneously. Thus, by combining them synergistic benefits are produced.

It has been found that certain specific nutrients inhibit, or block, the RANK/RANKL/NFKB signal pathway, thus markedly enhancing the effect of the known dietary mechanisms because of the impact on convergent pathways and the subsequently markedly enhanced effects.

The mechanism for the synergistic activity (blockade) against the RANK/RANKL/NFKB pathway involves activation of a novel signaling pathway called the AMP Kinase signaling pathway. AMP stands for Adenosine Mono Phosphate, a unit of energy; and Kinase refers to a specific enzyme activity. So, AMP Kinase (an enzyme), when activated, produces a decrease in activity of the RANK/RANKL/NFKB signaling pathway.

The nutrients that turn on, or increase, the activity of AMP Kinase and have been shown to improve bone metabolism and subsequent fat metabolism and nutrient partitioning include:

Hesperidin (from rinds of citrus fruits)

Berberine (from Coptis chinensis)

Epigallocatechin 3-Gallate (from green tea)

Resveratrol (from red grapes)

Curcumin (from Curcuma longa)

Ginsenosides (from Panax ginseng)

Gingerols (from fresh ginger)

Hispidulin (from snow lotus)

Quercetin (from many fruits)

Chronic metabolic acidosis has also been shown to impair insulin-signaling in a manner that results in a biochemical and hormonal situation that is referred to as “insulin resistance.” Insulin resistance plays a major role in the development of obesity, diabetes, vascular disease, and cardiovascular disease by its impact on lipid metabolism (such as producing deleterious impact on serum cholesterol and triglyceride (stored fat) levels, for example). In addition, chronic metabolic acidosis has been linked with increases in waist circumference and BMI (Body Mass Index—an indicator of obesity) thought to occur from related mechanisms. By combining what is taught in the related application for enhanced effectiveness of compositions taught in U.S. Provisional Patent Application Ser. No. 63/249,979, teaching the use of nutrients that buffer acid and inhibit the COX enzymes, with a combination of AMP Kinase activating nutrients, unexpected synergistic weight loss and metabolic benefits have been noted.

Activation of the enzyme AMP Kinase, in addition to its beneficial effects on bone metabolism, has been show to decrease fat accumulation and weight gain by increasing fat oxidation and glycogen storage and by diminishing fat synthesis and storage, thus beneficially enhancing nutrient partitioning. Since each of these above listed nutrients are activators of AMP Kinase, they would be associated with weight loss and the associated metabolic benefits related to that weight loss. In addition, activation of AMP Kinase produces improvements in glucose control and lipid metabolism, such as cholesterol and triglycerides. Hence, they, both individually and together, would enhance metabolic health and help maintain a healthy weight. Because they each augment AMP Kinase in a different fashion, and some do so directly while others do so indirectly, a unique and novel and unexpected synergism develops in their mutual actions. This produces a novel benefit not expected from a product or approach designed to enhance bone health.

In addition, the same nutrients that activate AMP Kinase also have additional and varied beneficial effects on related metabolic signaling pathways that further enhance the desired effects. The various pathways just mentioned are determined by the unique chemical composition and functionality of the chosen nutrients and their combined effects.

These nutrients, if consumed in varying formulations, amounts and ratios, may be combined with nutrients mentioned in the U.S. Pat. No. 6,579,866 that teaches how to turn on thermogenic (weight loss) cycles in the liver, which patent is incorporated herein by reference as if fully set forth herein. The AMP Kinase activators turn on a unique fat burning enzyme in the liver (Acetyl CoA Carboxylase) that potently accelerates fat burning. This would further increase the weight loss benefits of the carbohydrate futile cycles. In addition, AMP Kinase turns on UCP1 (uncoupling Protein 1) in brown fat cells. This burns fat calories and dissipates the energy released as heat. This provides an additional potent synergistic, weight loss effect. Novel synergistic benefits for weight loss and metabolic health would be expected because the aforementioned U.S. Pat. No. 6,579,866 teaches the use of nutrients to turn on thermogenic (calorie burning) carbohydrate futile cycles. This speeds up calorie burning. When combined with the benefits from AMP Kinase activation (decreased fat synthesis and increased fat burning (oxidation)) further synergy would ensue because they activate complementary calorie burning pathways. The nutrients that activate futile carbohydrate cycles include: Pyruvate, Aspartic acid, Chromium, Biotin, L-Carnitine. Additional formulations might include: Vitex agnus-castus because it suppresses appetite by stimulation of dopamine D2 receptors in the brain.

In sum, the patent extension and its formulations serve the following functions: (1) markedly and unexpectedly improve bone metabolism, decrease bone loss and enhance bone health, (2) produce unexpected benefits on weight loss and metabolic health, and (3) combine the benefits of both what is taught in the related technology described above and the novel improvements described above on both bone health and metabolic health/weight loss.

The invention herein has been described and illustrated with reference to the disclosed embodiments, including the combinations and permutations of the several nutrients, but it should be understood that the features and operation of the invention as described is susceptible to modification or alteration without departing significantly from the spirit of the invention. For example, the types and effective amounts of the various nutrients may be changed and or may be altered to fit specific applications. Additional similar nutrients may be uncovered that provide for addressing similar pathways that enhance bone health and reduce fat accumulation in the body. Accordingly, the specific embodiments described herein are for illustrative purposes only and the invention is not limited except by the following claims.

What is claimed is:

1. In an orally or parenterally administered composition for modulating nutrient partitioning in a human or animal so as to increase oxidation of fat, decrease storage of fat, enhance nutrient partitioning and increase storage of glycogen comprising an effective amount one or more of nutrients selected from the group consisting of Hesperidin, Berberine, Epigallocatechin 3-Gallate, Resveratrol, Curcumin, Ginsenosides, Gingerols, Hispidulin, Honokiol, Magnolol and Quercetin.

2. The composition as in claim 1 wherein the at least one of said nutrients components include at least one ingredient selected from the group consisting of hydroxycitric acid, carnitine, biotin, a gluconeogenic substrate, and, optionally, one or more of chromium, conjugated linoleic acid, coenzyme Q10, eicosapentaenoic acid, pyridoxine, alpha-lipoic acid, magnesium, and gymnema sylvestre.

3. In an orally or parenterally administered composition for diminishing AMP Kinase activity and modulating nutrient partitioning in a human or animal so as to increase oxidation of fat, decrease storage of fat, enhance nutrient partitioning and increase storage of glycogen comprising an effective amount one or more of nutrients selected from said composition comprising the following nutrients:
a) a first component selected from the group consisting of Quercetin, Curcumin, Resveratrol, Hispidulin and Hesperidin, each of which nutrients inhibit the synthesis of fat, block the storage of fat, suppress the differentiation of pre-adipocytes to mature fat cells, and decrease inflammation;
b) a second component selected from the group consisting of Berberine, Epigallocatechin 3-gallate (EGCG), Gingerols, Honokiol, each of which nutrients act to enhance thermogenesis by turning on enzymes that facilitate browning of white adipose tissue that speed up calorie burning; and
c) a second component selected from the group consisting of Ginsenosides and Magnolol for suppressing appetite,
said first second and third components each collectively provided in an effective amount for supporting and promoting healthy hydrogen ion balance, healthy calcium metabolism, healthy muscle metabolism, enhancing glycogen storage and enhancing nutrient partitioning, inhibiting the synthesis of fat and blocking the storage of fat in the body of said human being or animal.

4. A method of administering an effective amount of the composition of claims 1 and 3, comprising the steps of:
a) formulating the composition; and
b) administering the composition orally or parenterally.
